(19) United States
(12) Patent Application Publication
(10) Pub. No.: US 2007/0049814 A1
(43) Pub. Date: Mar. 1, 2007

(54) SYSTEM AND DEVICE FOR NEUROMUSCULAR STIMULATION

(76) Inventor: Philip E. Muccio, Columbus, OH (US)

Correspondence Address: MCNEES, WALLACE & NURICK LLC, 100 PINE STREET, P.O. BOX 1166, HARRISBURG, PA, 17108-1166, US

(21) Appl. No.: 11/466,637

(22) Filed: Aug. 23, 2006

Related U.S. Application Data
(60) Provisional application No. 60/710,802, filed on Aug. 24, 2005.

(51) Int. Cl. A61B 5/04

(52) U.S. Cl. 600/388

(57) ABSTRACT

A wearable neuromuscular stimulation and neuroprosthetic system and device for treating spinal cord injury, stroke, and other neurological conditions; and for the management of chronic pain. This invention provides a system for transcutaneous neuromuscular stimulation and typically comprises a wearable item that further includes a flexible, non-conductive material; at least one flexible, generally flat electrode attachable to or embedded within the wearable item; and a programmable electrical stimulation device connectable to the electrode(s). Each electrode typically includes a silver-impregnated or silver-treated material.

### CROSS-REFERENCE TO RELATED APPLICATIONS

This patent application claims the benefit of U.S. Provisional Patent Application Ser. No. 60/710,802 filed on Aug. 24, 2005 and entitled “Neuroprosthetic Systems and Systems for Therapeutic Muscle Stimulation and Pain Management”, the disclosure of which is incorporated by reference as if fully rewritten herein.

### TECHNICAL FIELD OF THE INVENTION

This invention relates generally to systems and devices for stimulating muscle tissue by electrical means, and more specifically to a system and series of garments for transcutaneous stimulation of muscle tissue for the treatment of spinal cord injury and/or chronic pain.

### BACKGROUND OF THE INVENTION

Victims of spinal cord injury may suffer from the loss muscle function in their upper and lower extremities, as well as from the loss of muscles that control posture and lower extremity circulation. If the spinal cord lesion is complete, or if the loss of neurological function is sufficient to prevent the patient from standing or walking and he or she is confined to a wheelchair, the patient may be prone to skin breakdown in the area on which he or she sits. The underlying bony prominences of the pelvis, which are the structures that bear the support of the body when sitting, are not well protected from pressure. Because they are thin and usually lack significant density, the gluteals and the hamstrings do not provide sufficient cushion and pressure distribution to guard bony structures from undue pressure and skin breakdown.

Decubitus ulcers are common in persons who sit for long periods of time and do not have the ability to alleviate seating surface pressures. Skin breakdown can force these individuals out of the wheelchair and into bed for long periods of time until the wound heals. Time spent in bed often represents a significant loss for the individual and family, and can result in the loss of a job, reduction in income, depression, and overall diminished quality of life. Many spinal cord injury patients become hospitalized and are forced to lie on special air-circulating beds that minimize pressure to the skin to promote microcirculation and healing. This can be extremely expensive, and such treatment is not always successful. Thus, many spinal cord injury patients with skin breakdown do not sufficiently heal, and they must undergo surgical procedures to close the wound(s). Such procedures are usually expensive and they may not prevent future skin breakdown. Furthermore, invasive surgical procedures may damage the nerve supply to the gluteal and hamstring muscles, thereby causing these muscles to become flaccid or incapable of voluntary or reflexive contraction. Flaccid muscle tends to become extremely thin, fibrous, and unable to provide underlying bones with padding and protection. Thus, there is a need for a system, method and/or device for assisting spinal cord injury patients with decubitus ulcers and other skin conditions that may result from prolonged sitting.

Muscles must contract frequently and with vigor to sustain their density, thickness and vascularization, and for purposes of oxygen uptake and metabolite removal. A spinal cord injury patient may have significant muscle atrophy because the signals from the brain are disrupted and the muscle cannot contract properly, if at all. Wheelchair bound individuals must take precautions to frequently unload the weight of the body because body weight can occlude the flow of blood to the skin and the underlying muscles. Without proper oxygen uptake, skin and muscle cells may undergo breakdown, wound formation and necrosis. Wheelchair bound individuals are susceptible to tissue breakdown because they are not able to stand. Standing is vital because it alleviates pressure on the buttocks and can provide valuable load and stress to the bones, range of motion and stretching to the ligaments and joints of the spine, hip, knee and ankle. Standing also provides the individual with the ability to reach for objects and to enjoy eye-to-eye contact with other standing individuals. Thus, there is a need for a system, method and/or device for assisting spinal cord injury patients with muscle contraction and standing, as well as with maintaining muscle density, thickness and vascularization

Spinal injury also often results in paralysis of the abdominal muscle. Over time, this leads to the patient's loss of an effective counterforce against gravity. The weight of the internal organs may apply pressure to the interior of the abdominal wall, and if the abdominal muscles are not able to oppose these forces, the muscles tend to shift generally downward and forward, thereby causing the abdominal wall to expand. Additionally, a quadriplegic or paraplegic usually loses the ability to exercise and expend energy from activity, especially the large muscle groups of the lower extremities, back and abdominal muscles themselves. The net gain of calories over time leads to an increase in the storage of adipose tissue underlying the abdominal wall. Together, the accumulation of adipose tissue and the force of internal organs unopposed by the patient's musculature, increase the ovoid shape of the abdominal wall leading to poor posture, diminished appearance, and other problems. Thus, there is a need for a system, method and/or device for assisting spinal cord injury patients with maintaining the strength of their abdominal muscles and maintaining decent posture.

### SUMMARY OF THE INVENTION

The following provides a summary of exemplary embodiments of the present invention. This summary is not an extensive overview and is not intended to identify key or critical aspects or elements of the present invention or to delineate its scope. This invention relates to wearable neuromuscular and neuroprosthetic systems and devices for treating spinal cord injury, stroke, and other neurological conditions; and for the management of chronic pain.

In accordance with one aspect of the present invention, a system for transcutaneous neuromuscular stimulation is provided. An exemplary embodiment of this system comprises a wearable item, i.e., a garment, that further includes a flexible, generally non-conductive fabric or material; at least one flexible, generally flat electrode attachable to or embedded within the wearable item; and a programmable electrical stimulation device connectable to the electrode(s). Each electrode typically includes a first fabric layer; at least one piece of silver-treated material in contact with the first fabric layer; a length of electrical wire, wherein a portion of the electrical wire has been de-insulated, and wherein the de-insulated portion of the wire is in contact with the piece of silver-treated material; a second fabric layer, wherein the second fabric layer covers the piece of silver-treated material and the de-insulated portion of the electrical wire; and a connector attached to the length of electrical wire opposite the de-insulated portion of the wire.

In accordance with another aspect of the present invention, a device for transcutaneous neuromuscular stimulation is provided. An exemplary embodiment of this device comprises a wearable item, i.e., a garment, wherein the wearable item further includes a flexible, generally non-conductive material; and at least one flexible, generally flat electrode attachable to or embedded within the wearable item. The electrode typically includes a first fabric layer; at least one piece of silver-treated material in contact with the first fabric layer; a length of electrical wire, wherein a portion of the electrical wire has been de-insulated, and wherein the de-insulated portion of the wire is in contact with the piece of silver-treated material; a second fabric layer, wherein the second fabric layer covers the piece of silver-treated material and the de-insulated portion of the electrical wire; and a connector attached to the length of electrical wire opposite the de-insulated portion of the wire, wherein the connector is adapted to receive electrical input from at least one programmable electrical stimulation device. This exemplary embodiment may also include at least one flexible, generally flat conductor located between and in contact with both the piece of silver-treated material and the de-insulated portion of wire. The flexible conductor typically includes silver-treated material. The silver-treated material of the electrodes and the conductors may include a protectively coating.

Additional features and aspects of the present invention will become apparent to those of ordinary skill in the art upon reading and understanding the following detailed description of the exemplary embodiments. As will be appreciated, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIG. 1 is a stylized front view of an individual wearing the neuromuscular stimulation/neuroprosthetic system of the present invention, wherein multiple embodiments of the wearable item are shown on a single human figure.

FIG. 2 is a rear perspective view of an exemplary embodiment of the neuromuscular stimulation/neuroprosthetic device of the present invention configured as a vest.

FIG. 3 is a rear view of an exemplary embodiment of the neuromuscular stimulation/neuroprosthetic device of the present invention configured as briefs.

FIG. 4 is a side view of an alternate embodiment of the briefs configuration of the neuromuscular stimulation/neuroprosthetic device of the present invention.

FIG. 5 is a front view of an exemplary embodiment of the neuromuscular stimulation device/neuroprosthetic of the present invention configured as a belt.

FIG. 6 is a rear view of an exemplary embodiment of the neuromuscular stimulation device/neuroprosthetic of the present invention configured as a belt.

FIG. 7 is a top view of an exemplary embodiment of the electrode component of the neuromuscular stimulation/neuroprosthetic device of the present invention.

### DETAILED DESCRIPTION OF THE INVENTION

Exemplary embodiments of the present invention are now described with reference to the Figures. Reference numerals are used throughout the detailed description to refer to the various elements and structures. For purposes of explanation, numerous specific details are set forth in the detailed description to facilitate a thorough understanding of this invention. It should be understood, however, that the present invention might be practiced without these specific details. In other instances, well-known structures and devices are shown in block diagram form for purposes of simplifying the description.

The present invention relates to wearable neuromuscular stimulation and neuroprosthetic systems and devices for: (i) treating spinal cord injury, stroke, and other neurological conditions; and (ii) for the management of chronic pain. A first general embodiment of this invention provides a system for transcutaneous neuromuscular stimulation. An exemplary embodiment of this system comprises a wearable item that further includes a flexible, generally non-conductive material; at least one flexible, generally flat electrode attachable to or embedded within the wearable item; and a programmable electrical stimulation device connectable to the electrode(s). Each electrode typically includes a first fabric layer; at least one piece of silver-treated material in contact with the first fabric layer; a length of electrical wire, wherein a portion of the electrical wire has been de-insulated, and wherein the de-insulated portion of the wire is in contact with the piece of silver-treated material; a second fabric layer, wherein the second fabric layer covers the piece of silver-treated material and the de-insulated portion of the electrical wire; and a connector attached to the length of electrical wire opposite the de-insulated portion of the wire. A second general embodiment of this invention provides a device for transcutaneous neuromuscular stimulation. An exemplary embodiment of this device comprises a wearable item, wherein the wearable item further includes a flexible, generally non-conductive material; and at least one flexible, generally flat electrode attachable to or embedded within the wearable item. The electrode typically includes a first fabric layer; at least one piece of silver-treated material in contact with the first fabric layer; a length of electrical wire, wherein a portion of the electrical wire has been de-insulated, and wherein the de-insulated portion of the wire is in contact with the piece of silver-treated material; a second fabric layer, wherein the second fabric layer covers the piece of silver-treated material and the de-insulated portion of the electrical wire; and a connector attached to the length of electrical wire opposite the de-insulated portion of the wire, wherein the connector is adapted to receive electrical input from a programmable electrical stimulation device. These exemplary embodiments may also include at least one flexible, generally flat conductor located between and in contact with both the piece of silver-treated material and the de-insulated portion of wire. The flexible conductor typically includes silver-treated material similar to or the same as the silver treated material included in the electrode. The treated material of the electrodes and the conductors may be protectively coated or otherwise treated for the purpose of protecting the electrodes and conductors and prolonging the useful life thereof.

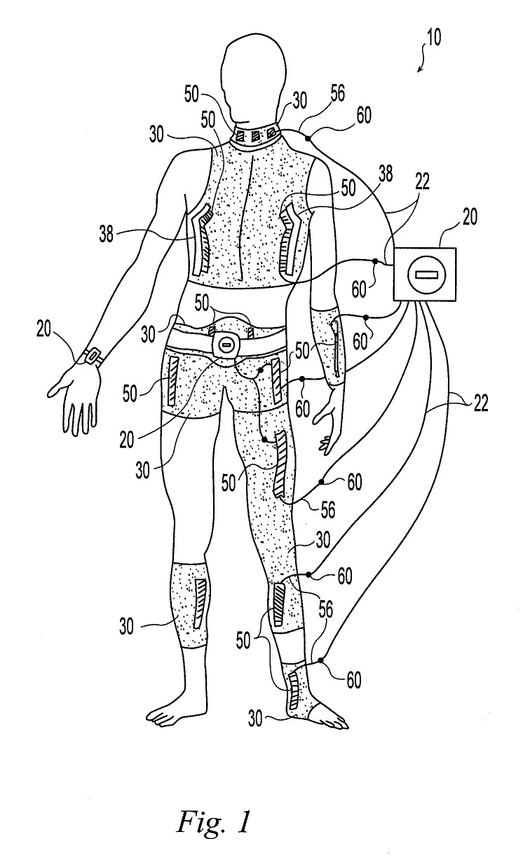

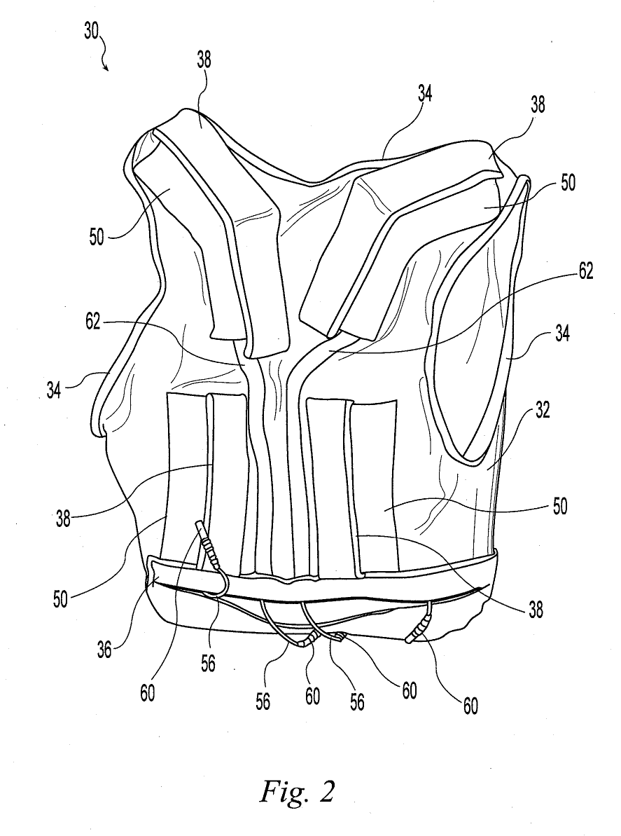

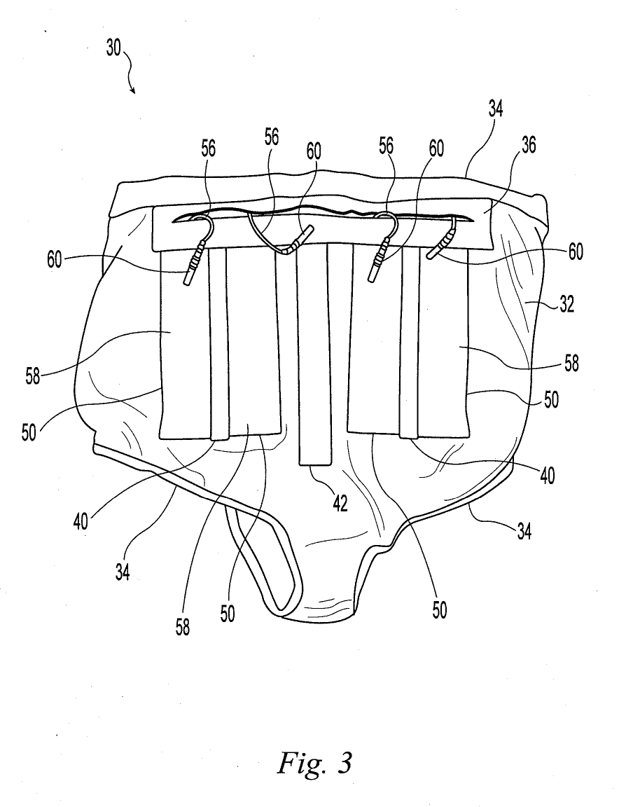

With reference now to the Figures, FIG. 1 illustrates multiple embodiments of the neuromuscular stimulation device of the present invention on a stylized human form. It should be understood that FIG. 1 is merely illustrative in nature, as it is highly unlikely that any one patient would wear all possible versions of the device at once. As shown in FIG. 1, an exemplary neuromuscular stimulation system 10 typically includes a programmable stimulator device 20 connected to or otherwise in communication with one or more wearable items 30. Programmable stimulator device 20 may be separate from the individual using system 10, or it may be worn directly on or around the waist, the wrist, or another body part. The wearable item 30 is a garment that includes multiple openings 34 and may be configured as any of a number of garment styles, including: a collar, vest, sleeve, shirt, belt, shorts, briefs, trousers, sock, or a suit (see also FIGS. 2-6). Combinations of these garments may also possible for certain applications. Wearable item 30 is typically made of a flexible, generally non-conductive material 32 such as lycra and/or spandex and may include one or more garment support members 40 and/or securing members 42 (see FIG. 3). Each wearable item also includes at least one, and usually a plurality of, electrodes 50 that are attached to or embedded within the fabric of the garment. Each electrode 50 delivers precisely controlled electrical energy to the user of the system transcutaneously, and each electrode may be situated on the top surface of material 32, on the bottom surface of material 32, or may simply be sewn into wearable item 30. A closeable gel pocket or reservoir 38 may also be included (see FIG. 2) with each electrode, particularly when the electrode 50 is situated on the top surface of material 32. Garment support members 40 function as stays or boning that help keep the garment from rolling up and help maintain physical distance between the electrodes 50. Securing members 42 serve as attachment points for an “elastic wrap around” device, which may be utilized to apply additional external pressure to the electrodes 50 for the purpose of maintaining complete or nearly complete contact with the skin of the wearer of the garment.

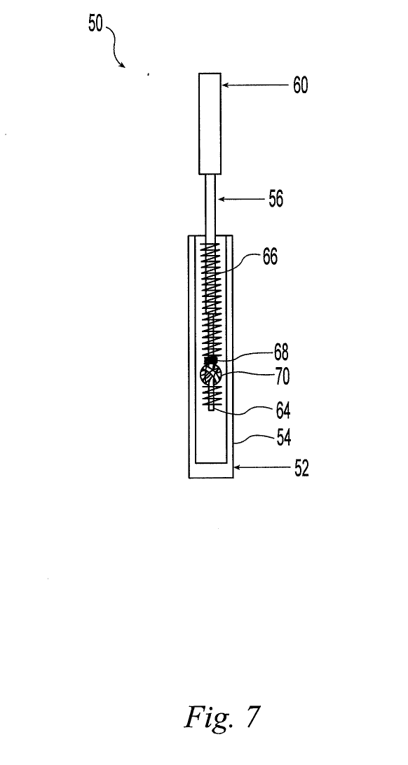

FIG. 7 illustrates an exemplary embodiment of an individual electrode 50, which may be any number of shapes and sizes. In this embodiment, electrode 50 includes a first fabric layer 52, which may be either an absorbent material or a water-barrier material. First fabric layer 52 is also “insulating” in that it minimizes the likelihood that either the wearer of the garment or a person who touches the garment will be inadvertently exposed to electric current. An electrically conductive, silver-treated material is then placed on the top surface of the first fabric layer 52. The term “silver-treated” refers to a fabric or material that has been coated with one or more layers of silver or that is woven from fibers that have been individually and coated with one or more layers of silver. As shown in the FIG. 7, an electrical wire 56 is attached to the silver-treated material 54 for the purpose of transmitting controlled electrical energy into and through the silver-treated material to the individual using wearable item 30. As shown in the Figure, a portion of electrical wire 56 has been de-insulated and tied into a retaining knot 70. The remaining de-insulated portion 64 of electrical wire 56 and a length of the insulated portion of electrical wire 56 are attached to silver-treated material 54 by loose stitching 66 and tight stitching 68. A second fabric layer 58, which covers the silver-treated material and de-insulated portion 64, is typically included (see FIG. 3); thus, the silver-treated material is typically sandwiched between two pieces of non-conductive fabric. In the exemplary embodiments described herein, the silver-treated material, which is commercially available in sheets or rolls, may be cut and sized according to the overall design of electrode 50. The silver-treated material is typically coated with at least two layers of an organic compound or other protective substance to prolong the life of the electrode on the body.

In the exemplary embodiment, at least one, and typically a plurality of electrodes 50 are in electrical communication with a programmable stimulator device 20. Programmable stimulator device 20 may be any of a number of devices, either off-the-shelf or custom designed and built, that are capable of delivering electric current to the electrodes 50 in a controllable and predictable manner. Programmable stimulator device 20 may include multiple channels, may be microprocessor-controlled, may be portable, and may include a transponder for wireless operation. As shown in FIG. 1, which depicts multiple alternate embodiments of this invention, electrical wires 56 are connected to lead wires 22 by connectors 60. Each connector 60 is typically a receptacle adapted to receive lead wire pins. In some embodiments of this invention, the electrodes 50 are in electrical communication with the programmable stimulator device 20 by wireless means; thus, connector 60 may be or may include a transceiver or other signal-receiving device.

In some embodiments of this invention, flexible conductors 62 connect the electrodes 50 to the electric wires 56. These flexible conductors are generally flat and include one or more layers of electrically conductive silver-treated material, which is the same as, or similar to the silver-treated material included in the electrodes 50. Inclusion of one or more flexible conductors 62 in or on wearable item 30 reduces the number of electrical wires 56, resulting in a more comfortable and less cumbersome garment. Also, reducing the number of electrical connections that the user (i.e., patient) must make before the system can be operated, simplifies the use of the system in general. Additionally, certain embodiments of wearable item 30 include zippered or otherwise closeable pockets 36, which are useful for storing/enclosing device wires 56 and connectors 60 so that these items are kept out of the way of the user of system 10.

As previously stated, the present invention is useful for treating spinal cord injury, stroke, and other neurological conditions; and for the management of chronic pain. Depending on the combination of the garment and the program running on programmable stimulator 20, therapeutic muscle stimulation, i.e., neuromuscular stimulation, may be achieved or neuroprosthetic effects may be achieved. Therapeutic muscle stimulation may prevent or reverse muscle disuse atrophy, reduce plasticity, increase local blood flow, improve range of motion, and prevent deep vein thrombosis. When partial voluntary control remains, neuromuscular stimulation may increase the strength of the involved muscle groups. Neuroprosthetic effects may provide functional restoration by allowing a muscle or group of muscles to contract on command or automatically to produce a desired action such as opening a hand. To use the present invention, a person suffering from spinal cord injury or other neuromuscular trauma or disorder simply places an appropriately configured (e.g., vest or shorts) wearable item 30 on their body, connects the electrodes 50 to the programmable stimulator 20, and runs a pre-programmed electrical stimulation routine. Depending on the placement of the electrodes 50 on or within the wearable item 30, an electrically conductive gel is placed either directly on the surface of first fabric layer 52 that contacts the skin of the user or within closeable gel reservoir 38, prior to the user placing wearable item 30 on their body.

Having generally described this invention, a further understanding can be obtained by reference to certain specific examples detailed below, which are provided for purposes of illustration only and are not intended to be all inclusive or limiting unless otherwise specified.

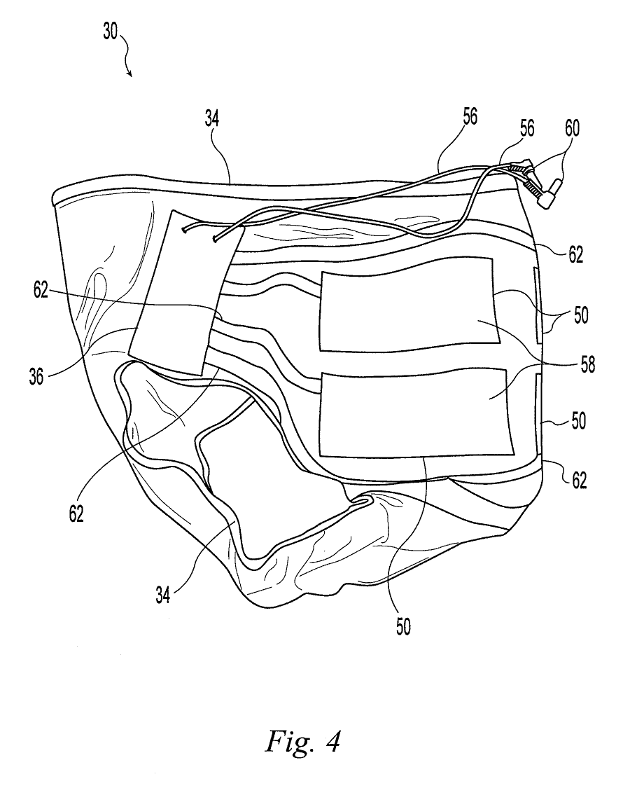

A more specific exemplary embodiment of the system and device of the present invention is the “MyoShorts and Multi-Mode (MM) Muscle Stimulator” combination (see FIGS. 3-4). This embodiment includes a lycra-spandex, electrode-embedded garment and a multi-channel microprocessor-controlled muscle stimulator that is worn on the body of the spinal cord injured patient throughout the day or for short durations such as 1-2 hours. This embodiment enables the user to select from a variety of menu options that deliver exercise options, standing, weight-shift and other muscle functionality. The “MyoShorts” can be worn for extended hours without a degradation of the contact between skin and electrode enabling patients to wear the device for many hours without having to reapply gel to the electrodes 50. The Multi-Mode (MM) Muscle Stimulator (programmable stimulator device 20) provides multiple programs from which to choose for purposes of posture (back extensor stimulation); improving sitting posture in the spinal cord; correcting spinal alignment problems i.e., scoliosis, kyphosis, and muscle imbalances; reciprocal stimulation to the gluteals and hamstrings left to right to alter seating pressures; reducing pressure; improving blood flow to the gluteal muscles and otherwise preventing skin breakdown in patients confined to wheelchairs; standing at will; pain control; circulation; and exercise.

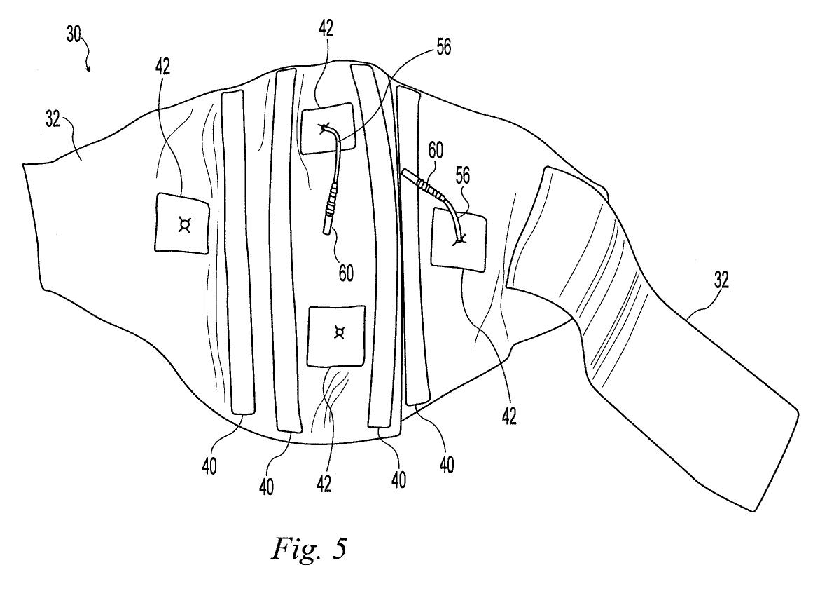

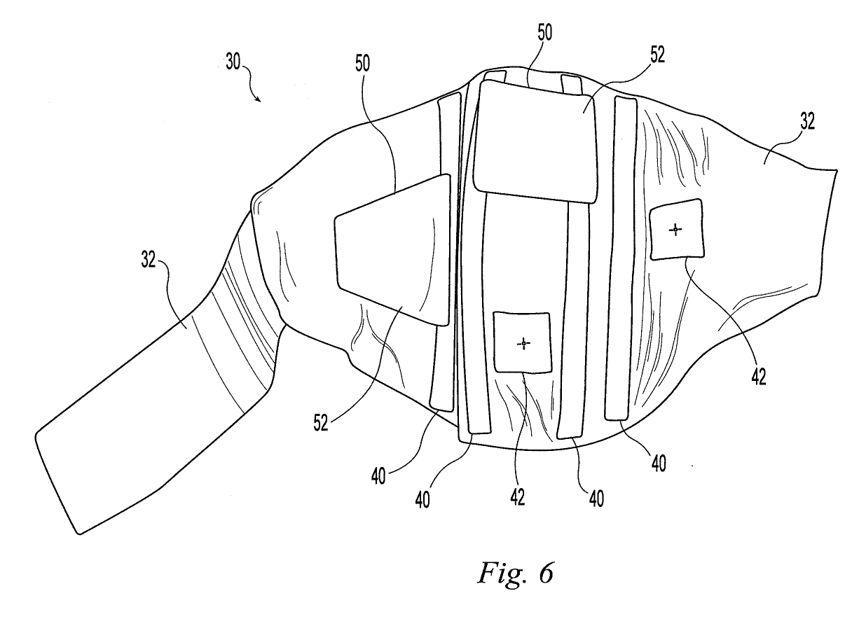

Another more specific exemplary embodiment of the system and device of the present invention is the “BioBelt-AB-4E” (see FIG. 5-6), which stimulates abdominal muscles. This embodiment provides an abdominal electrode belt that conforms to the pendulous shape of the typical quadriplegic or paraplegic having paralyzed abdominal muscles. The BioBelt-AB-4E is designed to maximize stretch of the fabric that comprises the front panel of the belt. Flexible, water-resistant and insulating material (e.g., Darlexx) allows the fabric to stretch in two dimensions to accommodate any convexity of the exterior abdominal wall. Tapering the front panel at specific locations on the belt creates an initial convexity and the use of detachable electrodes 50 (see FIGS. 5-6) permits the front panel to stretch as needed. Only a small section of the electrode 50, typically the center, is fixed to the front panel, either by Velcro, stitching, or other means.

Advantages of the neuromuscular stimulation and neuroprosthetic systems and devices of the present invention include: effective management of acute and chronic pain; effective management of sports-related injury; rehabilitation of muscle in orthopedic and sports injury; rehabilitation of damaged or paralyzed muscle; restoration of lost physiological processes in the neurologically-impaired; and restoration of function in the neurologically-impaired. Additionally, weak muscles can be strengthened to address the primary and secondary causes of pain; muscle tightness, contracture and spasticity can be reduced; muscle stimulation may release endogenous opiates (natural pain inhibitor); cortical recruitment and awareness of non-used muscle improves; muscle imbalances can be addressed; and local blood circulation can be increased.

Advantages of the design of the wearable item 30 and the electrode 50 include: the ability to accurately place the electrode over affected muscle groups; the patient may be able to place the electrodes without assistance; the electrodes do not typically wear out in a short period of time; the lead wires are minimized and the complexity of set-up is reduced; the electrode can be designed to cover a very large surface area; the device is wearable while sleeping to improve sleep loss stemming from pain, and can be worn throughout the day to deliver pain relief and muscle therapy; the electrodes maintain their contact with the patient more consistently than adhesive electrodes; the system conducts electricity through hair; and variances in body shape and size are easily managed.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the applicant's general inventive concept.

What is claimed:

1) A system for transcutaneous neuromuscular stimulation, comprising:
(a) a wearable item, wherein the wearable item further comprises a flexible, non-conductive material;
(b) at least one flexible, generally flat electrode attachable to or embedded within the wearable item, wherein the at least one electrode further comprises at least one piece of silver-treated, electrically-conductive material; and
(c) a programmable electrical stimulation device connectable to the at least one electrode.

2) The system of claim 1, wherein the wearable item further comprises a vest, belt, briefs, shorts, or combinations thereof.

3) A system for transcutaneous neuromuscular stimulation, comprising:
(a) a wearable item, wherein the wearable item further comprises a flexible, non-conductive material;
(b) at least one flexible, generally flat electrode attachable to or embedded within the wearable item, wherein the electrode further comprises:
(i) a first fabric layer;
(ii) at least one piece of silver-treated material in contact with the first fabric layer;
(iii) a length of electrical wire, wherein a portion of the electrical wire has been de-insulated, and wherein the de-insulated portion of the wire is in contact with the piece of silver-treated material;
(iv) a second fabric layer, wherein the second fabric layer covers the piece of silver-treated material and the de-insulated portion of the electrical wire; and
(v) a connector attached to the length of electrical wire opposite the de-insulated portion of the wire; and
(c) a programmable electrical stimulation device connectable to the at least one electrode by way of the connector.

4) The system of claim 3, further comprising a conductive gel in electrical communication with the at least one electrode, and wherein the conductive gel is substantially solid.

5) The system of claim 3, further comprising a conductive gel in electrical communication with the at least one electrode, and wherein the conductive gel is substantially liquid.

6) The system of claim 3, further comprising at least one flexible, generally flat conductor located between and in electrical communication with both the piece of silver-treated material and the de-insulated portion of wire, and wherein the flexible conductor further comprises silver-treated material.

7) The system of claim 3, wherein the wearable item further comprises at least one closeable pocket, and wherein each connector and a portion of the electrical wire to which the connector is attached may be contained within the closeable pocket.

8) The system of claim 3, wherein the wearable item further comprises at least one generally flat garment support member attached to or embedded within the wearable item.

9) The system of claim 3, wherein the wearable item further comprises a closeable conductive gel reservoir in contact with each electrode.

10) The system of claim 3, wherein the wearable item further comprises at least one securing member and at least one garment support member attached to or embedded within the wearable item.

11) The system of claim 3, wherein the wearable item further comprises a vest, belt, briefs, shorts, or a combination thereof.

12) The system of claim 3, wherein the flexible, non-conductive material of the wearable item further comprises lycra, spandex, or combinations thereof.

13) The system of claim 3, wherein the first fabric layer further comprises a water-barrier material or an absorbent material.

14) A device for transcutaneous neuromuscular stimulation, comprising:
(a) a wearable item, wherein the wearable item further comprises a flexible, non-conductive material;
(b) at least one flexible, generally flat electrode attachable to or embedded within the wearable item, wherein the at least one electrode further comprises:
(i) a first fabric layer;
(ii) at least one piece of silver-treated material in contact with the first fabric layer;
(iii) a length of electrical wire, wherein a portion of the electrical wire has been de-insulated, and wherein the de-insulated portion of the wire is in contact with the piece of silver-treated material;
(iv) a second fabric layer, wherein the second fabric layer covers the piece of silver-treated material and the de-insulated portion of the electrical wire; and
(v) a connector attached to the length of electrical wire opposite the de-insulated portion of the wire, wherein the connector is adapted to receive electrical input from a programmable electrical stimulation device.

15) The device of claim 14, further comprising at least one flexible, generally flat conductor located between and in electrical communication with both. the piece of silver-treated material and the de-insulated portion of wire, and wherein the flexible conductor further comprises silver-treated material.

16) The device of claim 14, wherein the wearable item further comprises at least one closeable pocket, and wherein each connector and a portion of the electrical wire to which the connector is attached may be contained within the closeable pocket.

17) The device of claim 14, wherein the wearable item further comprises at least one generally flat garment support member attached to or embedded within the wearable item.

18) The device of claim 14, wherein the wearable item further comprises a closeable conductive gel reservoir in contact with each electrode.

19) The device of claim 14, wherein the wearable item further comprises at least one securing member and at least one garment support member attached to or embedded within the wearable item.

20) The device of claim 14, wherein the wearable item further comprises a suit, vest, belt, collar, sock, sleeve, trousers, briefs, shorts, or combinations thereof.

21) The device of claims 14, wherein the flexible, non-conductive material of the wearable item further comprises lycra, spandex, or combinations thereof.

22) The device of claim 14, wherein the first fabric layer further comprises a water-barrier material or an absorbent material.
